(19) United States
(12) Patent Application Publication
(10) Pub. No.: US 2024/0058566 A1
(43) Pub. Date: Feb. 22, 2024

(54) STIMULUS GENERATOR

(71) Applicant: Tony Michael Guard, Union, KY (US)

(72) Inventor: Tony Michael Guard, Union, KY (US)

(21) Appl. No.: 18/235,928

(22) Filed: Aug. 21, 2023

Related U.S. Application Data
(60) Provisional application No. 63/474,498, filed on Aug. 22, 2022.

(51) Int. Cl. A61M 21/00; G16H 20/70

(52) U.S. Cl. CPC A61M 21/00; G16H 20/70; A61M 2021/005; A61M 2021/0027

(57) ABSTRACT

The presently disclosed subject matter provides a computer-implemented method and system for generating a stimulus. The method is generally implemented via a computer application installable on a user's computer/smart device. The stimulus generated by the application is displayed on the user's device screen and is generally a combination of outputs which are selected from words, text, images, sounds, and combinations thereof. The stimulus is intended to act as stimuli to the user in order to help drive new thoughts and ideas useful when trying to solve a problem, get inspiration, or be creative.

### TECHNICAL FIELD

The presently disclosed subject matter generally relates to a computer-implemented method for generating stimulus. The stimulus comprises texts, words, images, sounds, or combinations thereof.

### BACKGROUND OF THE INVENTION

To date, there are various computer-implemented methods which generate random words or phrases to assist with applications related to language and spelling. Most of these computer-implemented applications were developed for educational purposes. There are also computer-implemented applications that combine two words together to create potentially new words. Such computer-implemented applications are instrumental for creating new names for products or services, etc. Computer-implemented methods that help in orienting words for a better linguistic structure or for authentication when creating passwords are known in the art. Some methods, used as a means of education, which employ images are also known in the field. However, there is a need in the field for a computer-implemented application that provides a plurality of words, images, texts, sounds, or a combination thereof to provide a stimulus for the user.

### SUMMARY OF THE INVENTION

In accordance with the present invention, various embodiments of a computer-implemented method and system for generating stimuli are provided herein, wherein the computer-implemented method for generating stimuli comprises the steps of selecting at least two or more data group/s from a plurality of data groups, wherein each data group comprises a plurality of outputs, selecting at least one output from the plurality of outputs from each of the at least two or more data groups, wherein the at least one selected output from each of the at least two data groups is the stimuli.

In some of the embodiments of the computer-implemented method for generating stimuli, a user requests new stimuli; the new stimuli comprises selecting a different output from the plurality of outputs from each of the at least two data group/s.

In some of the embodiments of the computer-implemented method for generating stimuli, a user requests a new output instead of at least one of the selected outputs in the stimulus; the new output is selected from the plurality of outputs the same data group/s of the previously selected output/s.

In some of the embodiments of the computer-implemented method for generating stimuli, the output is selected from a group comprising text, words, images, and sounds.

In some of the embodiments of the computer-implemented method for generating stimuli, the at least two outputs are grouped in the at least one data group based on the at least two outputs meaning.

In some of the embodiments of the computer-implemented method for generating a stimulus, the user adds outputs as desired into the at least one data group/s.

In some of the embodiments of the computer-implemented method for generating a stimulus, the user creates at least one data group containing one or more output/s.

In some of the embodiments of the computer-implemented method for generating a stimulus, the user can access other users data group/s containing one or more output/s.

In some of the embodiments of the computer-implemented method for generating stimuli, the user determines which data groups are displayed on the user's device screen.

In some of the embodiments of the computer-implemented method for generating stimuli, the stimuli is displayed on a user's device screen for a predetermined amount of time.

In some embodiments the computer-implemented method for generating stimuli further comprising a signal to alert the user of the time remaining of the predetermined amount of time; wherein the signal is selected from a group comprising music, sounds, lights, or images.

In some of the embodiments of the computer-implemented method for generating stimuli, a user generated notation is stored by the user in the user device's memory or in a server memory.

In some embodiments the computer-implemented method for generating a stimulus further comprises wireless connection to the user's selected social media; enabling the user posts stimuli as desired.

In some embodiments, the presently disclosed system comprises:
a processor; and,
a memory; wherein the memory includes a storage to store plurality of data groups;
wherein the memory further comprises a stimuli generation engine, which, when executed on the processor, performs an operation that generates stimuli, the operation comprising:
generating two or more stimuli; and,
transmitting the stimuli to a user's device.

In some embodiments, the presently disclosed system comprises:
a user device comprising a processor and a memory; wherein the memory includes a storage to store plurality of data groups;
wherein the memory further comprises a stimuli generation engine, which, when executed on the processor, performs an operation that generates stimuli, the operation comprising:
generating two or more stimuli; and,
displaying the stimuli on the user's device screen.

### BRIEF DESCRIPTION OF DRAWINGS

Having thus described the presently disclosed subject matter in general terms, reference will now be made to the accompanying Drawings, which are not necessarily drawn to scale, and wherein:

FIG. 1 illustrates one example of the presently disclosed computer-implemented method for generating stimuli;

FIG. 2 illustrates another example of the presently disclosed computer-implemented method for generating stimuli;

FIG. 3 illustrates yet another example of the presently disclosed computer-implemented method for generating a stimulus; and,

FIG. 4 is a flow chart depicting an example of the system for presently disclosed computer-implemented method for generating stimuli.

### DETAILED DESCRIPTION OF THE DRAWINGS

The presently disclosed subject matter now will be described more fully hereinafter with reference to the accompanying Drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides a computer-implemented method and system for generating stimuli. Generally, the stimulus comprises at least two outputs. In some other embodiments, the stimulus comprises at least one output. In some embodiments, each output may be a word, a text, an image, a touch, or a sound. In some other embodiments, each output may be, without limitation, word/s, text/s, image/s, sound/s, touch, and combinations thereof. The stimulus generated is displayed on a user device's digital screen. Generally, each output is randomly selected from a data group which comprises outputs grouped together according to their common use meaning. The user decides which data groups are used for selecting outputs and thus generating stimulus. After reviewing a displayed stimulus, a user can choose to have other output/s displayed from his/her selected data group/s, i.e., create a different stimulus from the same data group/s. The stimulus is intended to act as a trigger to the user in order to help drive new thoughts and ideas useful when trying to solve a problem, get inspiration, or do creative writing.

In some embodiments, the presently disclosed computer-implemented method and system for generating stimulus comprises a computer program for carrying out the operations of the computer-implemented method for generating a stimulus as described and illustrated herein. The computer program for carrying out the operations of the computer-implemented method for generating stimulus is generally referred to herein as “Stimulus generator” or “Stimulus generator application”.

Computer program code for carrying out the operations for the present method may be written in any combination of one or more programming languages, including Java, Smalltalk, C++ or the like and conventional procedural programming languages, such as the “C” programming language or similar programming languages. The program code may execute entirely on the user's computer (all devices commonly known as “smart device”), for example, as a stand-alone software package, or partly on the user's computer and partly on a remote computer, or entirely on the remote computer or server. In the two latter scenario, the remote computer (referred to herein also as “server computer”) may be connected to the user's computer through any type of network, including a local area network (LAN) or a wide area network (WAN), or the connection may be made through the Internet using an Internet Service Provider. The term, or terms related to, “online connection” as used herein include, without limitation, all the above-listed connection types.

Aspects of the present method, system, and Stimulus generator application are described below with reference to flowchart and/or block diagrams illustrations. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer traditional in the art, such that the instructions, which execute via a computer, create means for implementing the functions/acts specified in the flowchart and/or block diagram block or blocks.

These computer program instructions may also be stored in a computer readable medium that can direct a computer, other programmable data processing apparatus, or other devices to function in a manner that implements the function/act specified in the flowchart and/or block diagram block or blocks. The computer program instructions may also be loaded onto a computer to cause a series of operational steps to be performed on the computer to produce a computer implemented process to allow the execution and functions/acts of the presently disclosed method and application on the computer and thus implement the functions/acts specified in the flowchart and/or block diagram disclosed herein.

For the purpose of the instant disclosure, a user's device may be any type of computer known and traditionally used in the art. Some unlimiting examples of computer applicable to the current disclosure include desktop computers, server computers, laptop computers, tablet computers, smart phones (“smart device”), and the like.

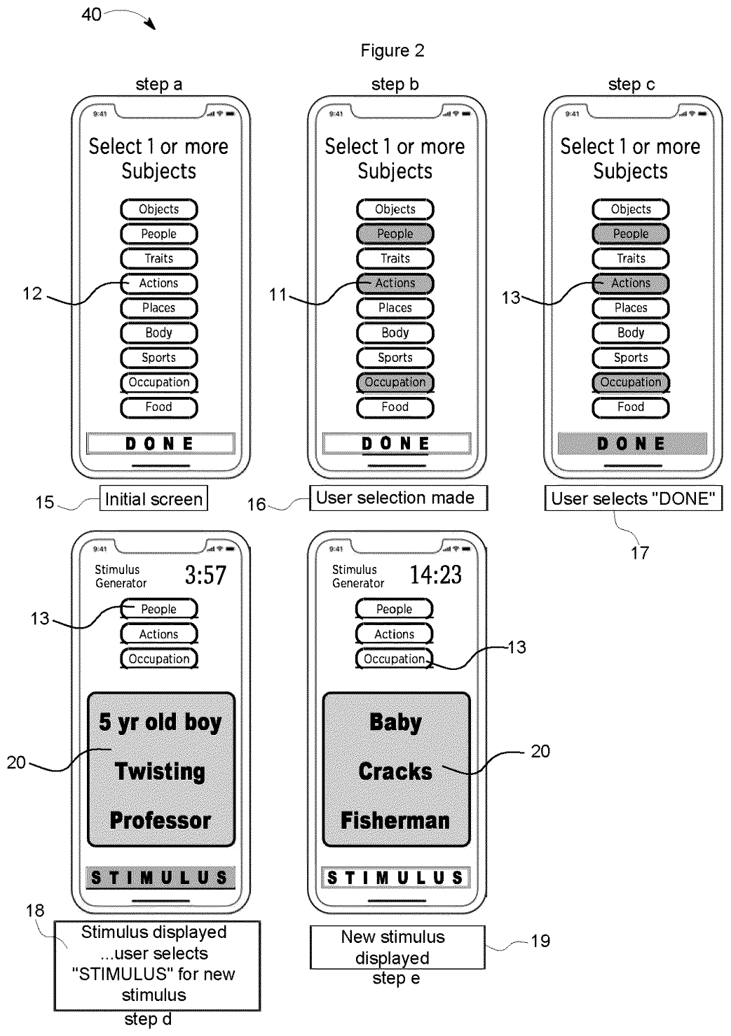

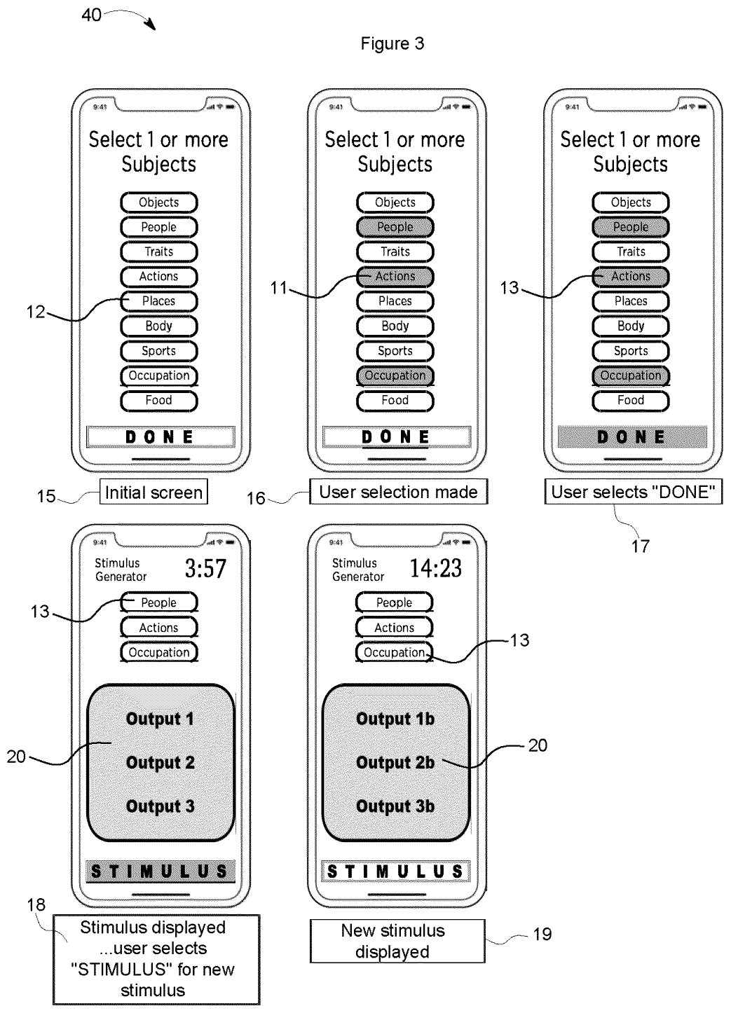

In some embodiments, the presently disclosed computer-implemented method and system for generating a stimulus includes a server computer system connected online to a plurality of users' devices. In some embodiments, the server computer includes computer program instructions for the implementation presently disclosed Stimulus generator application (40, FIGS. 2 and 3). The server computer may also include computer program instructions for enabling a user to download the computer program instructions for the implementation of presently disclosed Stimulus generator application onto the user's device. In some embodiments, the server computer includes a processor (not shown), such as a central processing unit (CPU), for executing computer program commands of any type known in the art. Any arrangement of a server computer may be used, as will be apparent to those of skill in the art upon reading the present description. In some embodiments, the server computer includes memory usable for storing data related to the Stimulus generator application and the Stimulus generator application user/s information. Such data includes, without limitation, data groups 12 and outputs 21 as classified into data groups 12. Such data may also include user/s' information such as user/s' customized data groups and outputs (will be further discussed below) and/or user/s' personal information. In some embodiments, each user's device communicates via the online connection to interact with the server computer.

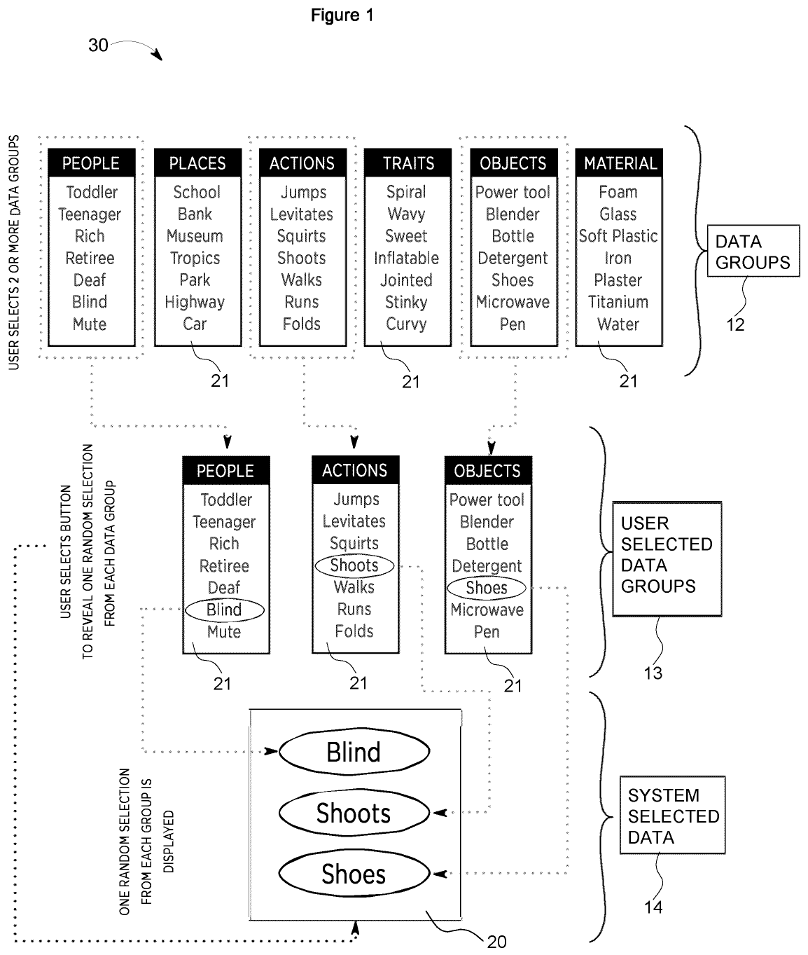

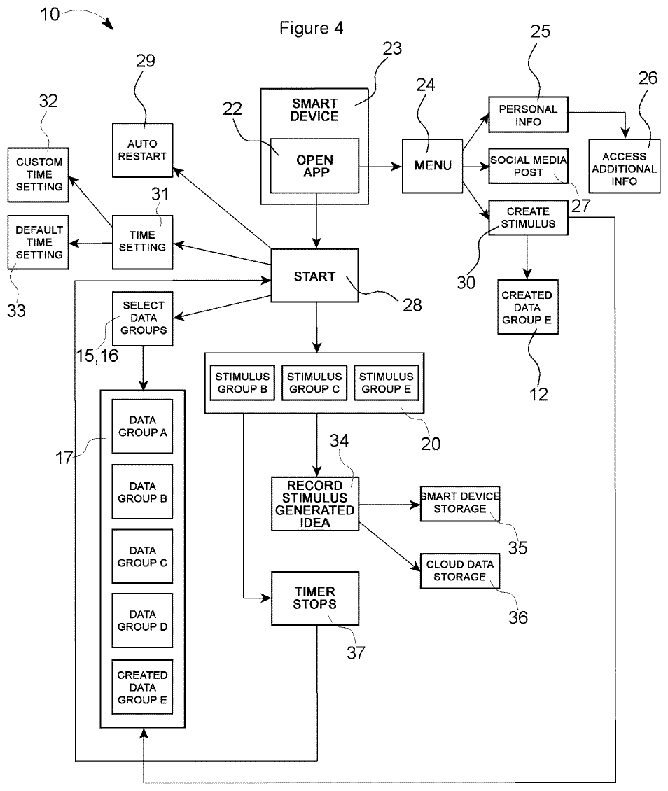

An example of the presently disclosed computer-implemented method 30 (also referred to herein as “method” or “the method”) for generating a stimulus 20 is depicted in FIG. 1. The method 30 includes a plurality of data groups 12 generally each devoted to a certain category. Examples of such categories are depicted in FIG. 1 and include people, places, actions, traits, objects, and materials. However, one skilled in the art will appreciate that the method may include an endless number of categories. Unlimiting examples of additional categories include shapes, food, colors, item of clothing, transportation means, etc. Data groups 12 are provided by the method 30, however, in some embodiments, may also be created by the user and be stored on the user's device 23 (FIG. 4). Each data group 12 includes at least one output 21. In some embodiments, each output 21 may be a word, a text, a sound, an image, or a touch. In some embodiments, the text may be a short sentence or a phrase. Outputs 21 provided by the method 30 are generally categorized into each specific data group 12 based on their commonly known semantic or visual meaning. For example, the semantic or visual information may specify that the output 21 denotes a place, an object, or an action.

A user may select at least one data group 12, in a preferred embodiment (shown) a user selects at least two data groups 12. The user selected data groups 13 are then displayed and the user can trigger a prompt, for example, by pressing a button, to reveal one selected output 21 from each of the user selected data group 13. Next, at least one output 21 is selected from each of the user selected data groups 13, these selected outputs 20, also referred to herein as “stimulus” are displayed to the user. The selection of the output/s which constitute the stimulus 20 is performed by the computer program for the implementation of the method 30 using any applicable algorithm known in the art, for example, an algorithm designed for a random selection of at least one item. The stimulus 20 may comprise any combination of words, texts, phrases, sounds, or images.

FIGS. 2 and 3 exemplify the Stimulus generator application 40 for performing the presently disclosed method 30 as implemented and used on a user's device 23 (depicted in FIG. 4), for example, a smart phone/smart device. In step a, an initial screen 15 is displayed depicting at least one data group 12, or a plurality of data groups 12 representing a variety of categories which are presented to the user. In step b, the user selects (user selection made 16) at least one data group 11 and these data groups 11 are highlighted/marked on the user's device 23 screen. The user can then make her/his selection final, for example, by pressing a button “done” (user selects “DONE” 17, step c), the highlighted data groups are now the user selected data groups 13. The Stimulus generator application 40 then functions (step d) to select at least one output 20 out of each selected data group 13 and display them, as a stimulus 20, to the user. The user decides at this point to stop and optionally, for example, store the generated stimulus 20 or generated idea 34 (will be further discussed below) or, alternatively, request 18 another stimulus (user selects “STIMULUS” for new stimulus) from the same selected data groups 13. The user may do so for example, by pressing a button, “Stimulus”, prompting the Stimulus generator application 40 to select a different at least two outputs 20 out of each of the selected data group 13 and display them, as a new stimulus 19, 20, to the user. In some embodiments, a user may request to change only some of the outputs 21 contained in a stimulus 20. For example, if a stimulus 20 consists of three outputs 21, the user may request a new output 21 in place of one or two of the current outputs 21 in the stimulus 20. For example, for the stimulus Baby Cracks Fisherman (step e of FIG. 2) a user may request to change the output 21 Baby, for example by pressing on the displayed word Baby, and a new output will be selected from selected data group 13 people, for example “Grandma”. Therefore, the next stimulus 20 displayed will be Grandma Cracks Fisherman.

In some preferred embodiments, the user is presented with at least two data groups 12 in step a. Each of the data groups 12 may comprise output/s 21 such as words, images, sounds, texts, or combination thereof. In some embodiments, the Stimulus generator application 40 provides the user with the option of recording sound/s. Further, in some embodiments, a user speech can be transcribed via the Stimulus generator application 40 and the resulting text may be stored in a data group 12 of choice as a new output 21. As another example, the text resulting from transcription of the user's speech may be stored by the Stimulus generator application 40 as a comment, for example a comment on a specific stimulus 20 that the user generated. The outputs 21 in a specific data group 12 relate to one another in meaning. For example, one data group may contain words representing “places”, such as desert, mountain, jungle, etc. The user can select at least one, two, three, or more data group/s 12. A user selecting three data groups is exemplified in step a. After selecting the data groups, the user presses a command on the smart device 23 that causes the Stimulus generator application 40 to finalize the selection and generate one selected output 21 from each selected data group 13 and display these outputs 20 simultaneously. In some embodiments, if three data groups 12 were chosen in step a, then there are three outputs 21 in the stimulus 20 displayed (one output 21 from each data group 13). If the user selected four data groups 13, then four outputs 21 constitute the stimulus 20 displayed, and so on. In some embodiments, more than one output 21 is selected from each of the user selected data groups 13, and all the selected outputs 21 are simultaneously displayed as a stimulus 20. In some embodiments, the user may determine how many outputs 21 are selected from each selected data group 13. For example, the user may determine that 2, 3, 4, 5, or more outputs will be selected from each selected data group 13 and thus the user determines the setting as desired.

In some embodiments, the stimulus generator application further comprises a timer 37 (FIG. 4), and each stimulus 20 generated is displayed for a predetermined amount of time, this is in order to keep a strategic pace on the stimulus/ideation effort. In some embodiments, the optional timer 37 setting is controlled by the user who may choose to have no time limitation to the display of the stimulus or set up a time limitation as desired. An indication of the amount of time remaining on the timer 37 may be achieved via the use of music, sound/s, light/s, or image/s, to name a few examples. Any of the indicators listed above may be built into the Stimulus generator application 40 or may originate from the user's device 23, such as, from the user's other applications, such as a music application.

FIG. 4 depicts an example of the presently disclosed system 10 for performing the presently disclosed method 30. In some embodiments, the system 10 further comprises a server computer. In some embodiments, the stimulus generator application 40 (also referred to herein as “the application” or “application”) is installed on a user's device 23, for example, when a user downloads the application 40 to the user's device 23 from a server computer. A user can then open the application 22 on his/her device 23 and start using it. A menu 24 screen is available to the user providing with the possibility of personalizing the application 40, for example, by creating a user profile comprising personal information 25 such as an avatar/photo, name, email address for notifications from the application 40, etc. The user can also choose to allow the application 40 access to additional information 26 such the user's contacts or connect the application 40 to the user's social accounts. Depending on the user preferences, the user may post his/her activity on the application 40 on social media 27, for example, a user may post a chosen stimulus 20 on a chosen social media platform and/or a stylus generated idea 34. Using the menu 24, a user may also personalize the application 40 in a manner related to the creation of a stimulus 27. For example, a user may create a new data group 12 (exemplified by “data group E”), not provided by the application 40. For example, a user who is interested in minerals may create a data group 12 related to this category and populate it with outputs 21. In some embodiments, a user can share the newly created data group 12 on social media and allow other users to help in populating it with outputs 21. A user can also personalize the application 40 by adding additional outputs 21 to existing data groups 12. In some embodiments, some, or all, of the data created or provided by the user is stored by the system 10.

In some embodiments, updates are provided and needed to update the application 40 which was downloaded to the user's device 23. These updates include updating the data groups 12, such as adding new data groups 12 and/or adding new outputs 21 to data groups 12. Updates may also remove data groups 12 or remove specific outputs 21.

Further, personal information 25 added by the user includes material which may be inspired by the stimulus/stimuli created by the user, such as notes, text, and documents. In some embodiments, this material can be stored in the user's device 23 memory, the application 40 provided memory 36, or a user's Provider memory via the application 40.

In some embodiments, a user can further customize the application 40 by setting an auto-start 29 and selecting the number of times different stimuli will be generated with each group of stimuli displayed for a specified amount of time. The auto-start 29 can start the application 40 automatically, for example every time the user start using the system 10. Alternatively, the application may auto-start based on any schedule set by the user. In some embodiments, a user can customize the application 40 time settings 31. For example, a user may set a custom time timer 37 as desired (custom time setting 32). Alternatively, a user may choose to use the application 40 default time setting 33 for the timer 37. In some embodiments, the custom time setting 32 and default time setting 33 are used to set up a desired amount of time for using the application 40. In some embodiments a user can choose to customize the time settings 31 and the auto-start option each time the user start 28 using the application to generate a stimulus 20.

After opening the application 22, 40, the user can start 28 the process of generating stimulus 20. In some embodiments, the user is directed to a screen 15, 16 which displays a plurality of data groups 12 and allows the user to select at least two data groups 12 from the said data groups 12. Once the user selects at least two data groups 12 and finalize the selection, the selected data groups 13 are displayed 17. The selected data groups 13 may include data groups 12 created by the user, for example, data group E. The application 40 than generates a stimulus 20, and upon a user request, may generate a plurality of stimuli 20 from the same user selected data groups 13. In some embodiments, all the stimuli generated in this manner are displayed simultaneously (shown). Consequently, a user may decide if to record/store 34 (such as, record by speech transcribed into text by the application 40 as described above) an idea inspired by the stimulus/stimuli generated. A user may store a stimuli, stimulus, or text via the application 40 in the user's device's 23 memory 35, or in a user Provider's memory or in the application's 40 provided memory 36.

In any of the embodiments described herein, verbal commands/sounds/images may be built into the application 40 to direct the user for the next screen/prompt or to encourage the user to use/continue to use the application 40 or to stimulate the user's idea process. For example, the application 40 may suggest links to videos which provide direction and examples of how to use the application. In another embodiment, the user is prompted with additional words, sounds, and images that may further promote new ideas while still viewing the stimuli generated from the data groups (such as, text “make it huge”, the sound of a police siren, a picture of a crowded sports venue, and so on).

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

By the term “substantially” it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide. For example, when using the term “substantially” herein it may be a value of at least 50%, at least 60%, at least 70%, at least 80%, at least 90%, or at least 99%, or any amount or range therebetween.

As one skilled in the art will appreciate, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

The present invention may be a system, a method, and/or a computer program product at any possible technical detail level of integration. The computer program product may include a computer readable storage medium (or media) having computer readable program instructions thereon for causing a processor to carry out aspects of the present invention. Any combination of one or more computer readable medium(s) may be used. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus or device.

A computer readable signal medium may include a propagated data signal with computer readable program code embodied therein, for example, in baseband or as part of a carrier wave.

Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Although the foregoing subject matter has been described in some detail by way of illustration and example for purposes of clarity of understanding, it will be understood by those skilled in the art that certain changes and modifications can be practiced within the scope of the appended claims.

1. A computer-implemented method for generating a stimulus, comprising:
one or more data groups each containing one or more outputs with each output relating to other outputs within the respective data group;
selecting at least one data group from a plurality of data groups, wherein each data group comprises a plurality of outputs;
selecting at least one output from the plurality of outputs from the data group, wherein the output is the stimulus.

2. The computer-implemented method for generating a stimulus of claim 1, wherein the new output/s (stimulus) is randomly selected from one or more data groups.

3. The computer-implemented method for generating a stimulus of claim 1, wherein the new output/s (stimulus) is based on a predetermined algorithm selection from one or more data groups.

4. The computer-implemented method for generating a stimulus of claim 1, wherein a user requests a new stimulus; the new stimulus comprises selecting a different output from the plurality of outputs from each of the at least one data group/s.

5. The computer-implemented method for generating a stimulus of claim 1, wherein a user requests a new output instead of at least one of the selected outputs in the stimulus; the new output is selected from the plurality of outputs the same data group/s of the previously selected output/s.

6. The computer-implemented method for generating a stimulus of claim 1, wherein the output is selected from a data group which may contain text, words, images, and/or sounds.

7. The computer-implemented method for generating a stimulus of claim 1, wherein the at least one output is grouped in the at least one data group based on the at least one output meaning.

8. The computer-implemented method for generating a stimulus of claim 1, wherein the user adds outputs as desired into the at least one data group/s.

9. The computer-implemented method for generating a stimulus of claim 1, wherein the two or more outputs are used by artificial intelligence to create an image representing the stimulus.

10. The computer-implemented method for generating a stimulus of claim 6, wherein the user creates at least one data group.

11. The computer-implemented method for generating a stimulus of claim 6, wherein the user determines which data groups output is displayed on the user's device screen.

12. The computer-implemented method for generating a stimulus of claim 6, wherein the stimulus is displayed on a user's device screen for a predetermined amount of time.

13. The computer-implemented method for generating a stimulus of claim 10, wherein a chosen stimulus or generated idea is stored by the user in the user device's memory or in a server memory.

14. The computer-implemented method for generating a stimulus of claim 11, further comprising a signal to alert the user of the time remaining of the predetermined amount of time; wherein the signal is selected from a group comprising music, sounds, lights, or images.

15. The computer-implemented method for generating a stimulus of claim 11, further comprising wireless connection to the user's selected social media; enabling the user posts of stimulus or stimulus generated idea(s) as desired.

16. A system comprising:
a processor; and,
a memory; wherein the memory includes a storage to store plurality of data groups of claim 1;
wherein the memory further comprises a stimulus generation engine, which, when executed on the processor, performs an operation that generates a stimulus, the operation comprising:
generating a stimulus; and,
transmitting the stimulus to a user's device.

17. A system comprising:
a user device comprising a processor and a memory; wherein the memory includes a storage to store plurality of data groups of claim 1;
wherein the memory further comprises a stimulus generation engine, which, when executed on the processor, performs an operation that generates a stimulus, the operation comprising:
generating a stimulus; and,
displaying the stimulus on the user's device screen.
